(19) United States
(12) Patent Application Publication
(10) Pub. No.: US 2012/0322977 A1
(43) Pub. Date: Dec. 20, 2012

(54) Micropatterned multifunctional inorganic nanoparticle arrays based on patterned peptide constructs

(75) Inventor: Eric H. Hill, Albuquerque, NM (US)

(73) Assignee: STC.UNM

(21) Appl. No.: 13/507,292

(22) Filed: Jun. 19, 2012

Related U.S. Application Data
(60) Provisional application No. 61/499,099, filed on Jun. 20, 2011.

(51) Int. Cl. C07K 17/14; C07K 1/113; B82Y 99/00; B82Y 25/00; B82Y 15/00

(52) U.S. Cl. 530/326; 530/300; 530/345; 530/327; 977/789; 977/953; 977/948; 977/932

(57) ABSTRACT

An inorganic nanoparticle array is self-assembled onto an unpatterned or patterned, peptide-functionalized substrate surface using peptide constructs comprising a substrate-binding peptide and a mineralization peptide.

### RELATED APPLICATION

This application claims benefits and priority of U.S. provisional application Ser. No. 61/499,099 filed Jun. 20, 2011, the entire disclosure of which is incorporated herein by reference.

### CONTRACTUAL ORIGIN OF THE INVENTION

This invention was made with government support under grant number W911NF 07-1-0079 awarded by Defense Threat Reduction Agency. The government has certain right in the invention.

### FIELD OF THE INVENTION

The present invention involves biomineralized inorganic nanoparticle arrays on micropatterned substrates and methods of fabricating them using peptide constructs comprising a substrate-binding peptide and a mineralization peptide.

### BACKGROUND OF THE INVENTION

There is a need for simply made arrays of inorganics for a variety of different applications and purposes, which have controllable features that would allow them to take on many different applications. Nanoparticles made of inorganic materials have many diverse properties which have a host of applications in many disciplines. For example, materials such as CaCO₃ and hydroxyapatite can form biomimetic structures that can be used for such applications as matrices for bone and tissue growth. Nanoparticle arrays which are made of CdS and some other materials can be used to improve the efficiency of solar thermal cells, through improved absorption of solar radiation. There are many applications involving silver and gold nanoparticles that can be used to generate surface plasmons, as well as many colors of photoluminescent quantum dots that could be used as sensors in conjunction with patterned protein arrays. In the present filing a method for simple and rapid fabrication of multifunctional nanoparticle arrays is disclosed.

Material binding peptides have been shown to successfully bind a specific material with binding affinities and equilibrium constants of dissociation shown to be similar to that of alkanethiols on gold (references 1, 2). In addition, peptides that can be used to mineralize salt from a solution into a nanoparticle have been discovered for a variety of different inorganic materials (references 4-6), such peptides being hereafter referred to as mineralization peptides.

### SUMMARY OF THE INVENTION

The present invention provides inorganic nanoparticle arrays that are self-assembled onto a peptide-functionalized substrate surface wherein the peptides are effective to bond to the substrate surface and also to precipitate (deposit) nanoparticles from an inorganics-containing medium subsequently brought into contact with the substrate. The peptides typically comprise peptide constructs comprising a substrate-binding peptide and a mineralization peptide. The nanoparticle arrays can comprise individual nanoparticles or nanoparticle structures (e.g. nanoparticle clusters, multi-layers, etc.) self-assembled on the substrate.

In an illustrative embodiment of the present invention, a substrate, such as silica or quartz, having an unpatterned or patterned surface is contacted with a first medium that includes the peptide constructs to form the peptide-functionalized substrate surface. The peptide-functionalized substrate surface then is contacted for a time with a medium containing inorganics, such as dissolved metal salts, for a time to precipitate or deposit inorganic nanoparticles on the substrate surface by virtue of self-assembly onto the peptide-functionalized substrate surface. The time of contact can be varied to control the size and/or number of nanoparticles deposited. The present invention thus envisions an inorganic nanoparticle array on a substrate surface comprising peptides bonded to the substrate surface and also bonded to the nanoparticles.

In this illustrative embodiment of the invention, gold nanoparticles can be deposited for fabrication of a biosensor embodying localized surface plasmon resonance (LSPR), CdS nanoparticles can be deposited for fabrication of high efficiency solar cells, FePt nanoparticles can be deposited for formation of biopatterned magnetic arrays that may find use for high-density memory devices, and intermixed FePt and gold nanoparticles can be co-deposited for fabricating magnetic quantum dots for purposes of illustration and not limitation. Moreover, practice of the invention can fabricate templates for directed mineralization of hydroxyapatite for bone support structures or biometric structures from silica and CaCO₃. Light-absorbing nanoparticle layers can be deposited pursuant to the invention to increase the efficiency of solar-power cells. Practice of the present invention thus is advantageous to provide a method for simple and rapid fabrication of multifunctional arrays of nanoparticles of various kinds.

Further advantages and features of the present invention will become apparent from the following detailed description taken with the following drawings.

### BRIEF DESCRIPTION OF THE DRAWINGS

FIGS. 1A, 1B, 1C, and 1D are diagrammatic views illustrating exemplary steps in practicing an illustrative method embodiment of the invention.

### DETAILED DESCRIPTION OF THE INVENTION

The present invention provides in an illustrative embodiment for the fabrication of biomineralized inorganic nanoparticle arrays on unpatterned or micropatterned substrates using multi-functional peptide constructs that comprise a substrate-binding peptide and a mineralization peptide. In one illustrative embodiment of the present invention, the substrate, such as silica or quartz or other suitable material, is not patterned when a substantially uniform nanoparticle coating or layer (as the nanoparticle array) is formed on the substrate surface. In another illustrative embodiment of the present invention, a substrate, such as silica, quartz or other suitable substrate material, has a substrate surface that first is patterned by any suitable patterning process to form a pattern corresponding to the eventual desired nanoparticle array configuration to be formed on the substrate surface. For purposes of illustration and not limitation, the substrate surface can be patterned using microcontact printing such as soft lithographic printing using octadecyltrichlorosilane (OTS) ink, alkanethiols, or any other suitable patterning material. Then, the unpatterned or patterned substrate surface is contacted with a first medium that includes multi-functional peptide constructs, which comprise a substrate-binding peptide and a mineralization peptide fused or joined together, in order to form a peptide-functionalized pattern corresponding to the eventual desired nanoparticle array configuration to be formed on the substrate surface. The peptide-functionalized substrate surface then is contacted with a second medium containing inorganics, such as dissolved metal salts, for a time to precipitate or deposit inorganic nanoparticles on the substrate surface by virtue of self-assembly onto the peptide-functionalized substrate surface. The time of contact can be varied to control the size and/or number of nanoparticles deposited on the substrate surface.

For purposes of further illustration and not limitation of the present invention, useful peptides can include fused peptide constructs of the following:

1. Quartz-Binding Peptide—LPDWWPPPQLYH

2. Gold Mineralization Peptide—AYSSGAPPMPPF

3. FePt Mineralization Peptide—HNKHLPSTQPLA

4. CdS Mineralization Peptide—GDVHHHGRHGAEHADI

wherein peptides 1-4 can be used to create genetically-fused peptide structures with a minimum of two repeats of a mineralization peptide (e.g. at least one of peptide 2-4) fused to a minimum of two repeats of a material binding peptide (e.g. peptide 1). This basic form of the fused peptide construct will be able to bind to a selected substrate material (e.g. silica or quartz) and then be placed in an appropriate second medium (inorganics-containing medium) for the precipitation of minerals to form nanoparticle arrays. The second medium can comprise a salt solution that correlates to the peptide used for mineralization, e.g. FeCl₂ and H₂PtCl₆ for mineralization of FePt nanoparticles using a mineralization domain with peptide 3 above.

More complex variations of this peptide construct can involve the use of proteins with genetically-fused mineralization and binding peptide domains, as well as the use of other types of functional peptides, such as gelation peptides. These peptide constructs will initially be fused at the N-terminus, but molecular dynamics (MD) studies can be used to assess other possible permissive sites when proteins and enzymes are used in the constructs. The multifunctionality of these more complex possible peptides lends the ability to use the arrays for a wide variety of applications.

In addition to the fusing of other proteins and functional peptides into the arrays, the ability to make stable nanoclusters of two different inorganic materials would be of great scientific value. For example, if peptides 3 and 2 above were both used as mineralization peptides, then the production of nanoparticles with both minerals would take place. These types of dually functional nanoparticles could be used in a broad array of applications. One example could be directing nanoparticles through magnetism for some delivery of capsules bound to the gold of the cluster for medical applications.

In addition to the peptide sequences discussed, there are a plethora of peptides that can be used for selective binding of substrates as well as peptides that can be used for the mineralization of inorganics out of solution into defined nanoparticle structures. The use of phage display techniques can also be used to find new peptides for a particular material binding affinity or the ability to mineralize nanoparticles of a desired composition from salt solution.

The quartz-binding peptide LPDWWPPPQLYH and its synthesis are described by Wei, J. H.; Kacar, T.; Tamerler, C. Sarikaya, M. Ginger, D.S., in Small 2009, 5, pp. 689-693, the teachings of which are incorporated herein by reference to this end. The gold (Au) mineralization peptide AYSSGAPPMPPF and its synthesis are described by Slocik, J. M. et al. in Small 2009, 5, pp. 689-693 and by Chen, C. L. et al. in J. Am. Chem. Soc.l 2008, 130, pp. 13555, the teachings of both of which are incorporated herein by reference to this end. The FePt mineralization peptide HNKHLPSTQPLA and its synthesis are described by Reiss B. D. et al. in NanoLett.l 2004, 4, pp. 1127, the teachings of which are incorporated herein by reference to this end. The CdS mineralization peptide GDVHHHGRHGAEHADI and its synthesis are described by Peelet, B. R. et al. in Acta Biomater. 2005, 1, pp. 145, the teachings of which are incorporated herein by reference to this end.

The peptides of interest can then be made through the use of a peptide synthesizer, or also through molecular biology techniques of protein and peptide expression. The molecular biology techniques can allow the use of proteins bound to peptide sequences for more advanced applications, and if only the peptide is needed it could be cleaved from the protein using restriction enzymes. The possibilities enabled by this technique would allow for creation of a variety of materials such as biomimetic structures, ferromagnetic alloys, multifunctional catalysts, and other advanced materials. In addition, the fabrication using peptide sequences lends to much simpler and greener chemistry than the current processes involving self-assembled monolayers and the attachment of fabricated nanoparticles onto surfaces using means such as alkanethiols on gold and Biotin-Streptavidin interactions.

Alternately, the fused peptide constructs can be synthesized by custom peptide suppliers or by use of recombinant plasmids commercially available from suitable suppliers. This would involve the transfection and ligation of the recombinant plasmids into a target bacteria that would then produce the peptides.

The present invention also envisions using genetically engineered peptides with variations in the number of repeating units of the binding and mineralization peptides. In addition, the placement of inert polymer or peptide spacers between the mineralization and binding domains is envisioned in the present invention. The present invention also envisions use of synthetic peptides known as peptoids in lieu of, or in addition to, the peptides discussed above. Useful peptides according to the present invention and as recited in the claims include, but are not limited to, the peptides discussed above including the synthetic peptides known as peptoids.

The unpatterned or patterned substrate surface is contacted with a first medium that includes multi-functional peptide constructs comprising a substrate-binding peptide and a mineralization peptide of the type described above or others in order to form a peptide-functionalized pattern corresponding to the eventual desired nanoparticle array configuration to be formed on the substrate surface. For purposes of illustration and not limitation, the peptide-containing first medium can comprise a pH-buffered aqueous solution of the peptides of interest. Contact typically is achieved by immersing the patterned substrate surface in the peptide-containing first medium for an appropriate time.

Then, the peptide-functionalized substrate surface is contacted with (e.g. immersed in) a second medium containing inorganics, such as dissolved metal salts, particular to the mineralization domain used, for a time to precipitate or deposit inorganic nanoparticles on the substrate surface by virtue of self-assembly onto the patterned, peptide-functionalized substrate surface. For purposes of illustration and not limitation, the inorganics-containing second medium can comprise precursor mineralization solutions which are aqueous or organic (organic solvents may require use of synthetic peptides known as peptoids.) and which contain inorganics that include, but are not limited to, dissolved metal salts. For purposes of further illustration and not limitation, a solution containing HAuCl₄ (e.g. 0.1M HAuCl4 aqueous solution) can be used to deposit gold nanopartcles; a solution containing FeCl₂ and H₂PtCl₆ (e.g. 0.075 M FeCl₂+0.025 M H₂PtCl₆ aqueous solution) can be used to deposit FePt nanoparticles; a solution containing CdCl₂ and Na₂S (e.g. 0.05 M CdCl₂ aqueous solution equilibrated for 12 hours to which is added 0.05 M Na₂S) can be used to deposit CdS nanopartcles; and a solution containing HAuCl₄, FeCl₂ and H₂PtCl₆ (e.g. 0.05 M HAuCl₄+0.05 M FeCl₂+0.0125 M H₂PtCl₆ aqueous solution) can be used to deposit clusters of intermixed FePt nanoparticles and Au nanoparticles.

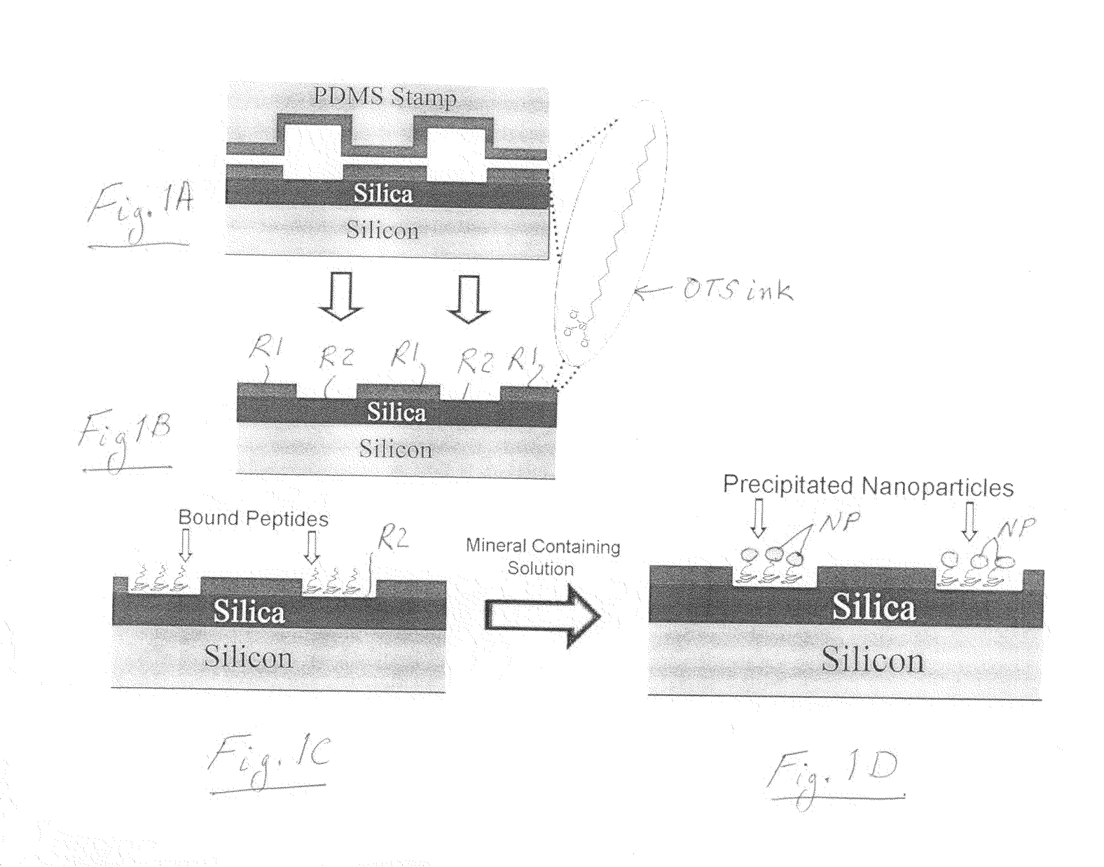

FIGS. 1A through 1D illustrate exemplary steps in practicing an illustrative method embodiment of the invention wherein in a first step, FIGS. 1A and 1B, a silica layer on a silicon chip is patterned by soft lithography using a PDMS (polydimethylsiloxane) stamp as shown to apply octadecyltrichlorosilane (OTS) ink (or other suitable patterning material) in a desired pattern wherein the applied pattern has ink-covered regions R1 and substrate-exposed (non-inked) regions R2. The substrate-exposed regions R2 define a pattern corresponding to the eventual desired nanoparticle array configuration to be formed on the substrate surface. The OTS ink is illustrated schematically in FIGS. 1A and 1B. After the OTS ink is applied, the substrate can be washed with toluene, then ethanol, then 18MΩ distilled water, and dried using pressurized nitrogen gas.

FIG. 1C illustrates the second step that involves bonding suitable peptide constructs to the exposed-substrate regions R2 by, for example, immersing the patterned substrate surface for a time in a peptide-containing first medium that can comprise for purposes of illustration and not limitation a 10 mM Tris-HCl (pH 7.5) buffer containing purified peptides of interest. The substrate surface can be immersed for a time (e.g. 16 hours or more or less) to form a peptide-functionalized pattern corresponding to the eventual desired nanoparticle array configuration to be formed on the substrate surface. Fused peptide constructs can be used to this end as described above. After the substrate surface is peptide-functionalized, the substrate can be washed with buffer and distilled water, and dried using pressurized nitrogen gas.

FIG. 1D illustrates the third step involving precipitating or depositing nanoparticles on the substrate surface by virtue of self-assembly onto the peptide-functionalized substrate surface. The precipitated nanoparticles NP are illustrated schematically in the figure as circles attached onto the upper end terminus of the peptide constructs, which are bound at the other end terminus to the substrate surface as schematically shown. Precipitation (deposition) of nanoparticles onto the substrate surface is achieved by immersing the peptide-functionalized substrate surface in the mineral-containing solution (second inorganics-containing medium) of the type described above. The immersion time can be varied to vary the size and/or number of the nanoparticles deposited on the substrate surface. After the nanoparticles are deposited, the substrate can be washed with distilled water and dried using pressurized nitrogen gas.

It should be noted that OTS patterning step is optional and not necessary for practice of the invention. If the OTS patterning step is omitted, the nanoparticle array will comprise a substantially uniform layer of nanoparticles on the substrate surface. However if one practicing the invention chooses to micropattern the surface, one would subject the substrate to a pattern removal treatment at some point in the processing to remove the pattern using one of many conventional pattern removal methods.

### EXAMPLE

The following example is offered to further illustrate practice of the invention without limiting the invention in any way:

Gold nanoparticle arrays for use in localized surface plasmon resonance (LSPR) applications can be formed using a polypeptide sequence consisting of five repeat units of the quartz-binding peptide 1 connected through the N-terminus to three repeat units of the gold mineralization peptide 2. After the optional patterning step of FIGS. 1A, 1B as described above, the substrate surface is immersed in an aqueous solution containing the polypeptides and allowed to stand for 24 hrs. The peptide-functionalized substrate then is placed in an aqueous bath of 0.1 M HAuCl₄ buffered at pH 7 for 12 hours. The time in the metal salt solution can be varied to change the final size of the nanoparticles formed. The resulting gold-nanoparticle coated silica surface can be used for a variety of LSPR applications, from more common uses as SPR reflectivity measurements used to detect molecular adsorptions and enhancement of Raman scattering measurements to more recent applications such as LSPR waveguides. One common application that could greatly benefit from this technology using gold nanoparticles on silica is the enchancement of light-absorption of solar cells due to the strong absorption bands in visible light exhibited by noble metals such as gold.

Although the present invention has been described in connection with certain illustrative embodiments, those skilled in the art will appreciate that the invention is not so limited and that modifications and changes can be made thereto within the scope of the invention as set forth in the appended claims.

References, which are incorporated herein by reference:

1. Kacar, T.; Zin, M. T.; So, C.; Wilson, B.; Hong Ma, Gul-Karaguler, N.; Jen, K. -Y. A.; Sarikaya, M.; Tamerler, C.; Biotechnology and Bioengineering 2009, 103(4)

2. Wei, J. H.; Kacar, T.; Tamerler, C.; Sarikaya, M.; Ginger, D. S. Small 2009, 5, 689-693.

3. Slocik, J. M.; Stone, M. O.; Naik, R. R. Small 2005, 1, 1048.

4. Chen, C. L.; Zhang, P. J.; Rosi, N. L.; J. Am. Chem. Soc. 2008, 130, 13555.

5. Reiss, B. D.; Mao, C. B.; Solis, D. J.; Ryan, K. S.; Thomson, T.; Belcher, A. M. Nano Lett. 2004, 4, 1127.

6. Peelle, B. R.; Krauland, E. M.; Wittrup, K. D.; Belcher, A. M. Acta Biomater. 2005, 1, 145.

I claim:

1. A method of forming an inorganic nanoparticle array, comprising contacting a peptide-functionalized substrate surface and an inorganics-containing medium to deposit inorganic nanoparticles on the peptide-functionalized-substrate surface.

2. The method of claim 1 wherein the peptide-functionalized substrate surface comprises peptide constructs comprising a substrate-binding peptide and a mineralization peptide.

3. The method of claim 1 wherein the peptide-functionalized substrate surface comprises synthetic peptides.

4. The method of claim 1 wherein the substrate comprises silica or quartz.

5. The method of claim 2 including patterning the substrate surface or not and contacting the patterned substrate surface with a peptide-containing medium.

6. The method of claim 5 wherein the peptide-containing medium comprises an aqueous medium containing the peptide constructs.

7. The method of claim 5 wherein the substrate surface is patterned by lithography.

8. The method of claim 1 wherein the inorganics-containing medium comprises a metal salt solution.

9. The method of claim 7 wherein the metal salt solution includes dissolved HAuCl₄, FeCl₂, H₂PtCl₆, CdCl₂, and/or Na₂S.

10. The method of claim 1 wherein nanoparticles comprising gold are deposited on the substrate surface.

11. The method of claim 1 wherein nanoparticles comprising CdS are deposited on the substrate surface.

12. The method of claim 1 wherein nanoparticles comprising FePt are deposited on the substrate surface.

13. The method of claim 1 wherein nanoparticles comprising intermixed FePt nanoparticles and Au nanoparticles are co-deposited on the substrate surface.

14. The method of claim 1 wherein the peptide-functionalized substrate surface and the inorganics-containing medium are contacted by immersing the substrate surface in the inorganics-containing medium.

15. An inorganic nanoparticle array on a substrate surface, comprising peptides bonded to the substrate surface and also bonded to the nanoparticles.

16. The array of claim 15 wherein the peptides comprise peptide constructs comprising a substrate-binding peptide and a mineralization peptide.

17. The array of claim 15 wherein the peptides comprise synthetic peptides.

18. The array of claim 15 wherein the substrate-binding peptide comprises LPDWWPPPQLYH.

19. The array of claim 15 wherein the mineralization peptide comprises AYSSGAPPMPPF; HNKHLPSTTQPLA; and/or GDVHHHGRHGAEHADI.

20. The array of claim 15 which is a biosensor, solar cell, magnetic array, and/or magnetic quantum dot.

21. The array of claim 15 which a substantially uniform layer on the substrate surface.
